(19) United States
(12) Patent Application Publication
(10) Pub. No.: US 2012/0197050 A1
(43) Pub. Date: Aug. 2, 2012

(54) PRODUCTION OF HYDROCARBON FUELS FROM PLANT OIL AND ANIMAL FAT

(76) Inventors: CHANDRASHEKHAR H. JOSHI, Bedford, MA (US); Michael Glenn Horner, West Roxbury, MA (US)

(21) Appl. No.: 13/362,314

(22) Filed: Jan. 31, 2012

Related U.S. Application Data
(60) Provisional application No. 61/462,381, filed on Feb. 1, 2011.

(51) Int. Cl. C07C 9/22; C25B 3/10; C07C 1/207; C07C 11/02; C07C 9/15

(52) U.S. Cl. 585/16; 585/324; 585/310; 205/415

(57) ABSTRACT

The present invention relates to fuel compositions and methods of making the same. The fuel compositions include hydrocarbon derived from a biological source selected from plant oil, animal fat and combinations thereof. The hydrocarbon and the fuel compositions are at least substantially oxygen-free. In particular, the fuel compositions are useful in cold temperature environments and as aviation fuel.

This patent application claims priority to U.S. Provisional Patent Application Ser. No. 61/462,381 which was filed in the United States Patent and Trademark Office on Feb. 1, 2011.

### FIELD OF THE INVENTION

The invention relates to fuel compositions and methods of making the same. These fuel compositions are at least substantially oxygen-free and useful, in particular, in cold temperature environments and as aviation fuel.

### BACKGROUND OF THE INVENTION

Global climate change is causing a shift in the sources of energy from fossil fuels to more sustainable and renewable resources, such as biodiesel. However, in cold climates, such as in temperate or polar regions of the world (including a significant portion of the United States, Canada, northern Europe and northern Asia), biodiesel fuels tend to solidify rendering inoperable engines that use it.

Furthermore, for aircraft, the energy densities available from batteries, fuel cells and other portable sources are not sufficient. Aviation fuel, such as jet fuel, is generally a specialized type of petroleum-based fuel used to power an aircraft and is generally of a higher quality than fuel used for ground transportation. Aviation fuel is designed to remain liquid at cold temperatures as found in the upper atmosphere where aircraft fly. Aviation fuels can include alkane hydrocarbons, such as paraffins; alkenes; naphthenes and other aromatics; antioxidants; and metal deactivators. Known aviation fuels include jet fuels, such as JP-5, JP 8, Jet A, Jet A-1, and Jet B. Aviation requires a high energy dense liquid fuel to achieve the speeds and distances airplanes can deliver today. Jet fuel has the highest volumetric energy density of liquid fuels, such as ethanol, butanol, bio-kerosene, and biodiesel.

There is a need in the art to develop hydrocarbon fuel compositions that can be a direct replacement for diesel fuel, home heating oil, and jet fuel that does not solidify in cold temperature environments for use in homes, ground transportation vehicles, and aircrafts. Further, it is desirable for these fuel compositions to satisfy requirements for use as aviation fuel and to be derived from a sustainable resource.

### SUMMARY OF THE INVENTION

In one aspect, the invention provides a fuel composition including a hydrocarbon derived from a biological source selected from the group consisting of plant oil, animal fat and combinations thereof and wherein each of the hydrocarbon and the fuel composition is at least substantially free of oxygen.

In another aspect, the invention provides a method for preparing a fuel composition. The method includes reacting a compound derived from a biological source selected from the group consisting of plant oil, animal fat and combinations thereof, with water to form free fatty acid; subjecting the free fatty acid to Kolbe electrolysis in the presence of an electrolyte, and removing an oxygen-containing carboxyl group from the free fatty acid to form a hydrocarbon.

### BRIEF DESCRIPTION OF THE DRAWINGS

The invention can be further understood by referring to the drawings which represent certain embodiments of the invention.

FIG. 1 is flow diagram of the process of the invention and three different configurations for employing the process in accordance with certain embodiments of the invention.

FIG. 2 is a chemical structure diagram to show a hydrolysis reaction of triglyceride into free fatty acids and glycerol in accordance with certain embodiments of the invention.

FIG. 3 is a chemical reaction diagram wherein Kolbe electrolysis is used to convert free fatty acid into linear hydrocarbons in accordance with certain embodiments of the invention.

FIG. 4 is a chemical structure diagram to show olefin metathesis, acid-catalyzed hydrolysis and Kolbe electrolysis reactions to produce a fuel composition from jatropha oil in accordance with certain embodiments of the invention.

### DETAILED DESCRIPTION OF THE INVENTION

The invention relates to hydrocarbon-containing fuel compositions and methods of making the same. These fuel compositions are at least substantially oxygen-free and made from sustainable plant oils, animal fats and mixtures and combinations thereof. These fuel compositions can be used in a wide variety of applications. In particular, the fuel compositions can be employed as a cold weather fuel for use in ground transportation vehicles, such as trucks, automobiles, railroads, and the like, and as an aviation fuel for use in aircrafts, such as airplanes, helicopters, and the like. Further, the fuel compositions can be used as a replacement for heating oil to heat houses and the like.

Suitable plant oils can be selected from a wide variety known in the art, such as soybean, jatropha, camelina, waste cooking oils, and other seed crops. Table 1 shows non-limiting examples of sources of plant oil including food and non-food crops which are known in the art and suitable for use in certain embodiments of the invention and the oil yield for these sources.

TABLE 1

| Crop | litres oil/ha | US gal/acre |
|---|---|---|
| corn (maize) | 172 | 18 |
| cashew nut | 176 | 19 |
| oats | 217 | 23 |
| lupine | 232 | 25 |
| kenaf | 273 | 29 |
| calendula | 305 | 33 |
| cotton | 325 | 35 |
| hemp | 363 | 39 |
| soybean | 446 | 48 |
| coffee | 459 | 49 |
| linseed (flax) | 478 | 51 |
| hazelnut | 482 | 51 |
| euphorbia | 524 | 56 |
| pumpkin seed | 534 | 57 |
| coriander | 536 | 57 |
| mustard seed | 572 | 61 |
| camelina | 583 | 62 |
| sesame | 696 | 74 |
| safflower | 779 | 83 |
| rice | 828 | 88 |
| tung oil | 940 | 100 |
| sunflower | 952 | 102 |
| cocoa (cacao) | 1026 | 110 |
| peanut | 1059 | 113 |
| opium poppy | 1163 | 124 |
| rapeseed | 1190 | 127 |
| olive | 1212 | 129 |
| castor bean | 1413 | 151 |
| pecan nut | 1791 | 191 |
| jojoba | 1818 | 194 |
| jatropha | 1892 | 202 |
| macadamia nut | 2246 | 240 |
| brazil nut | 2392 | 255 |
| avocado | 2638 | 282 |
| coconut | 2689 | 287 |
| oil palm | 5950 | 635 |

In general, it is known in the art that biodiesel fuel (“biodiesel”) has characteristics and properties that make it unattractive for use in cold weather environments. At low temperatures, certain molecules within biodiesel can agglomerate into solid particles. As a result, normally translucent biodiesel appears cloudy. The highest temperature at which the biodiesel begins to agglomerate or cloud is referred to as the cloud point. The cloud point is an important characteristic of fuels which are used in internal combustion engines and jet engines because the presence of solid or agglomerated particles can cause fuel pumps and injectors to clog rendering the engines inoperable. The cloud point for some known biodiesel products are as follows: 0° C. for canola; 1° C. for soybean; −6° C. for safflower; 1° C. for sunflower; −2° C. for rapeseed; 13° C. for jatropha; and 15° C. for palm. Aviation fuels known in the art have very low cloud points. The cloud points of various fossil fuels suitable for use as aviation fuels are as follows: 0° C. for ULS diesel; −40° C. for Jet A; −47° C. for JP-8; and −40° C. for ULS kerosene. For aviation fuels, a low cloud point is needed because the fuel must remain liquid at high altitude where temperatures can be well below zero. For ground transportation fuels, a low cloud point is important because when ground vehicles are used in cold weather environments, the fuel must remain liquid at relatively low temperatures.

The invention includes a process for making hydrocarbon fuel from plant oil and/or animal fat. The hydrocarbon fuel can include linear hydrocarbon, branched hydrocarbon and mixtures thereof. The hydrocarbon fuel is at least substantially free of oxygen (e.g., oxygen-free). The process includes hydrolysis and Kolbe electrolysis. The hydrolysis can include acid-catalyzed hydrolysis or base-catalyzed hydrolysis. In certain embodiments, the process can further include olefin metathesis. These reactions are known in the art. Further, known procedures for carrying out these reactions can be used in the process of the invention.

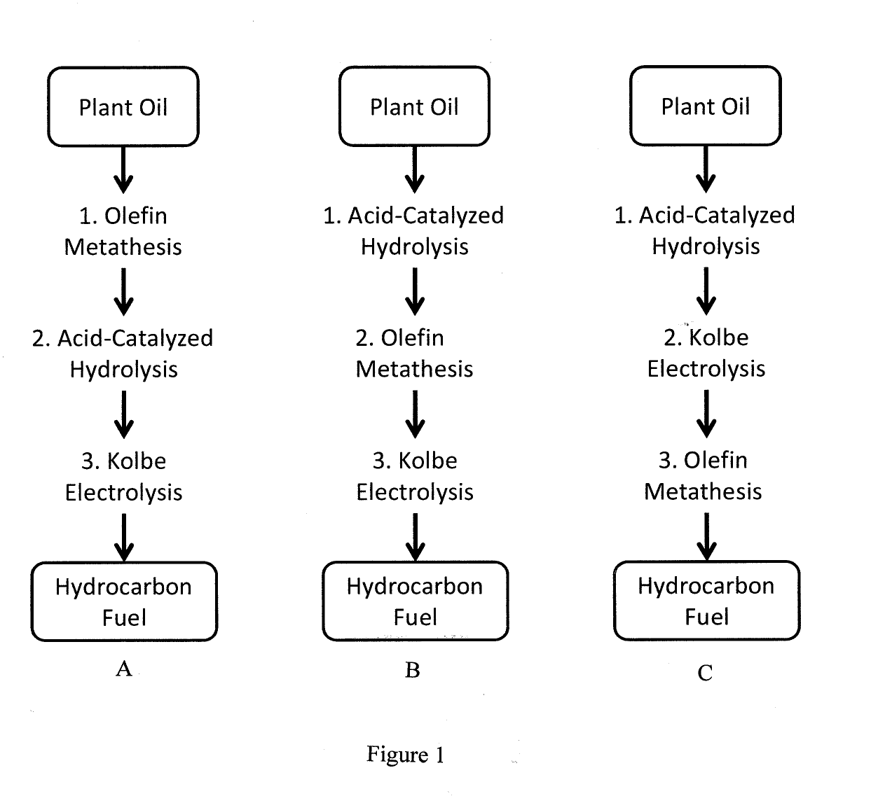

In accordance with certain embodiments of the invention, plant oil and/or animal fat can be used to produce hydrocarbon fuel by employing acid- or base-catalyzed hydrolysis and Kolbe electrolysis. In accordance with certain other embodiments of the invention, plant oil and/or animal fat can be used to produce hydrocarbon fuel by employing acid- or base-catalyzed hydrolysis, Kolbe electrolysis and olefin metathesis. When employed, olefin metathesis can be performed prior to the hydrolysis and Kolbe electrolysis or in-between the hydrolysis and Kolbe electrolysis or after both the hydrolysis and the Kolbe electrolysis. FIG. 1 shows various configurations for combining hydrolysis, Kolbe electrolysis and olefin metathesis to produce hydrocarbon fuel from plant oil. As shown in FIG. 1, configuration A includes subjecting the plant oil to olefin metathesis, then an acid-catalyzed hydrolysis, followed by Kolbe electrolysis. In configuration B, the plant oil is subjected to acid-catalyzed hydrolysis, then olefin metathesis, followed by Kolbe electrolysis. Configuration C identifies that the plant oil is subjected to acid-catalyzed hydrolysis, then Kolbe electrolysis, followed by olefin metathesis.

In accordance with certain embodiments, the invention can include olefin metathesis of plant oil with ethene (i.e., ethenolysis) or other lower alkene, such as propene, hydrolysis of the triglyceride esters in the oil to produce free fatty acid, and Kolbe electrolysis to remove the oxygen-containing carboxyl group, resulting in hydrocarbon or mixtures thereof having a low cloud point, such that the hydrocarbon is suitable for use as biodiesel in a variety of applications including cold temperature environments and aviation. In certain embodiments, branched hydrocarbons can be produced, for example, by use of 1,1-di-substituted alkenes, such as isobutylene in the metathesis reaction.

Hydrolysis is a known process that includes reacting plant oil or animal fat with water to break down the plant oil or animal fat into free fatty acid and glycerol. Optionally, a catalyst can be employed in the reaction. Further, the reaction can include the application of heat to accelerate the reaction.

The catalyst for use in the hydrolysis reaction can be selected from a wide variety known in the art to promote the reaction including acids and bases. The use of basic catalysts can produce carboxylic acid salts which are soaps and can function as surfactants. These soaps present processing challenges for product isolation and therefore, acid-catalyzed hydrolysis is preferred when free carboxylic acids are the desired product. In certain embodiments, the reaction pH is kept below the pKₐ of the product acid such that the product can segregate from the aqueous phase, and facilitate product isolation.

The acid catalyst for use in the hydrolysis reaction can be selected from a wide variety known in the art. Non-limiting examples include, but are not limited to, sulfuric acid, hydrochloric acid and mixtures thereof. It is known in the art to use solid or heterogeneous catalysts, e.g., Lewis acids, and microwaves for direct heating with excellent results in the hydrolysis of triglyceride. See, for example, Matos et al, J. Mol. Catalysis B: Enzymatic (72)1-2, pp 36-39, 2011. In a preferred embodiment, a solid catalyst is employed since it facilitates separation of the catalyst from the products upon completion of the reaction.

Suitable solid catalysts for use in the invention can be selected from those known in the art. Selection of a particular solid catalyst can depend on at least one of the following properties: surface area, pore size, pore volume and active site concentration on the surface of the catalyst. A wide variety of known solid catalysts can be used for the production of free fatty acids. Non-limiting examples can include, but are not limited to, zirconium oxide (zirconia), titanium oxide (titania), vanadium phosphate and mixtures thereof. Additional solid catalysts can be found in related literature, such as Zabeti, M. et al., Fuel Processing Technology, 90 (2009) p 770-777 and Ngaosuwan, K., et al., Ind. Eng. Chem. Res. 48 (2009) p 4757-4767 and Zubir, M. I. and S. Y. Chin, J. Applied Sci., 10 (2010) 2584-2589. In certain embodiments of the invention, methanol can be used in the hydrolysis reaction. In certain other embodiments, the methanol can be replaced with water.

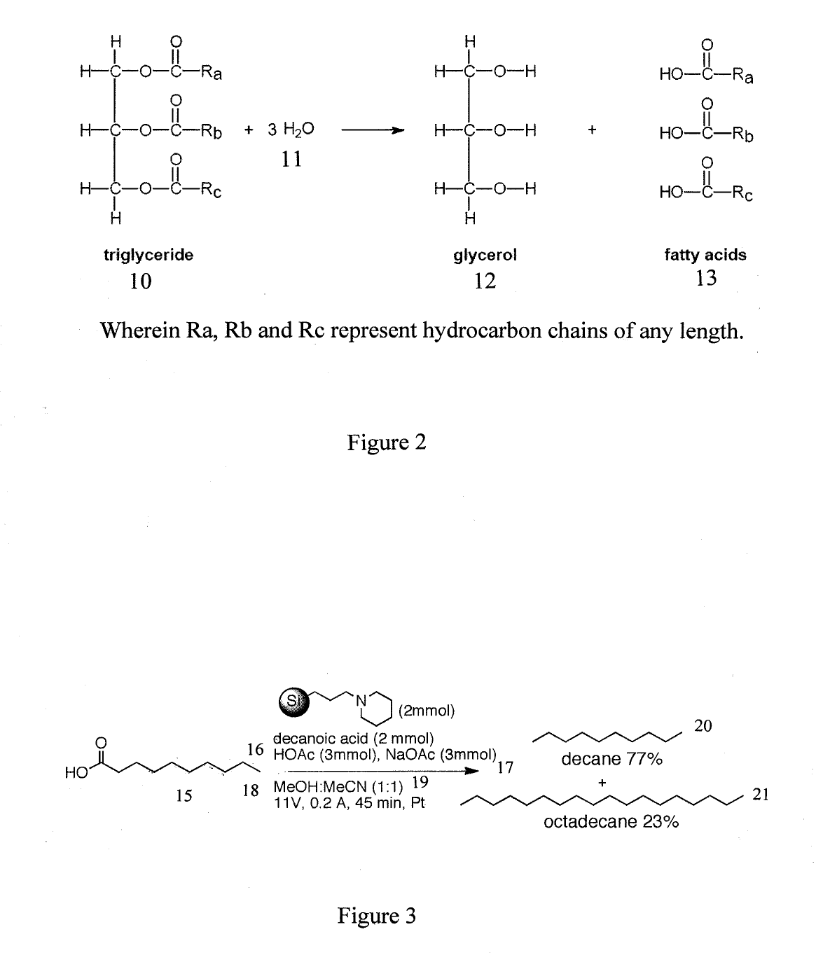

FIG. 2 shows a hydrolysis reaction in accordance with certain embodiments of the invention. As shown in FIG. 2, triglyceride 10 is reacted with water 11 to produce glycerol 12 and fatty acids 13. Triglyceride is the basic component of plant oils. In this reaction, triglyceride 10 includes substituents Rₐ, R_{b}, and R_{c} which represent hydrocarbon chains of any length.

The free fatty acids contain an even number of carbon atoms, from 4 to 36, bonded in an unbranched chain. Most of the bonds between the carbon atoms are single bonds. In certain embodiments, wherein all of the bonds are single bonds, the free fatty acid is said to be saturated because the number of atoms attached to each carbon atom is a maximum of four. In certain other embodiments, wherein some of the bonds between adjacent carbon atoms are double bonds, the free fatty acid is unsaturated. Without intending to be bound by any particular theory, when there is only one double bond, it is usually between the 9th and 10th carbon atom in the chain, where the carbon atom attached to the oxygen atoms is counted as the first carbon atom. If there is a second double bond, it usually occurs between the 12th and 13th carbon atoms, and a third double bond is usually between the 15th and 16th.

Kolbe electrolysis is a reaction to electrochemically oxidize carboxylic acids to produce alkanes, alkenes, alkane-containing products, alkene-containing products and mixtures thereof. The reaction is known to proceed through radical intermediates to yield products based on dimerization of these radicals, such that a n-carbon acid will give an alkane and/or alkene of length (2n−2) carbons along with two carbon dioxide molecules. In certain embodiments, the electrolysis reaction can be conducted in accordance with known processes and procedures, such as but not limited to the disclosure in Kurihara, H. et al, Electrochemistry, 74 (2006) 615-617. In the Kolbe electrolysis, only the carboxyl groups participate in the reaction and any unsaturation that may be present in the fatty acid chain is preserved in the final product. FIG. 3 shows a Kolbe electrolysis reaction in accordance with certain embodiments of the invention. As shown in FIG. 3, decanoic acid 15 is reacted in Kolbe electrolysis with acetic acetate 16, sodium acetate 17 and co-solvents methanol 18 and acetonitrile 19, with a silica gel-supported base 22, to produce decane 20 and octadecane 21.

The chain length of the product can be controlled by selection of feedstock and by providing an opportunity for heterocoupling between different sized acid chains. In the context of Kolbe electrolysis, heterocoupling is the reaction between two different carboxylic acids that results in an unsymmetrical product. Heterocoupling has been previously described in the art, such as by Levy, P. F.; Sanderson, J. E.; Cheng, L. K J. Electrochem. Soc., 1984, 131, 773-7 which investigated the coupling of mixtures of low molecular weight acids. In principle, heterocoupling of decanoic acid with acetic acid using this process yields decane. Heterocoupling of palmitic acid, found in soybean, jatropha and many other oils, with acetic acid can yield hexadecane. Lauric acid which is found in coconut oil, can be heterocoupled with acetic to yield dodecane. Hexadecane is very similar in composition to petroleum-based diesel fuel and dodecane is similar in composition to kerosene. Thus, in certain embodiments, hexadecane can be used as a sustainable fuel substitute for petroleum-based diesel fuel and dodecane can be used as a sustainable fuel substitute for kerosene.

In certain embodiments, when acetic acid and higher molecular weight fatty acids are placed in the Kolbe solution, both heterocoupling and homocoupling reactions can occur, and can lead to the production of very large homocoupled alkanes and/or alkenes and homocoupled product from acetic acid (e.g., ethane), which can result in a low yield of the desired heterocoupled product. Without intending to be bound by any particular theory, it is believed that to achieve higher yield of lower molecular weight oils, a chain transfer agent can be employed. In general, chain transfer agents are used to limit the length of carbon chains in radical polymerization reactions. A number of molecules contain hydrogen atoms that are readily removed by free radicals to yield a particularly stable species. Non-limiting examples of suitable chain transfer agents include hydroquinones, thiols, ethers, tertiary amines, and mixtures thereof. Hydroquinones may result in a radical which is stable such that it may be considered as inactive with regard to processes such as radical polymerizations. The use of other transfer agents may result in a radical that can participate in further reactions, thereby remaining kinetically active.

In certain embodiments, wherein chain transfer agents are used in Kolbe electrolysis, the radical chain transfer agents may terminate the intermediate alkyl radicals before they can dimerize. For this purpose, a chain transfer agent that is not easily oxidizable under the conditions of the Kolbe electrolysis may be selected. Thus, in certain embodiments, it is contemplated that hydroquinones, ethers, amines, and thiols may not be effective because they can be oxidized to new species which are no longer effective chain transfer agents. In certain other embodiments, an alcohol, such as but not limited to isopropanol, may be an effective chain transfer agent because it can contribute a hydrogen atom to yield a protonated ketyl radical that can 1) oxidize to acetone, 2) dimerize to give pinacol, or 3) couple with an (n−1) carbon alkyl fragment to yield a modest length alcohol. The tertiary alcohol so formed can be easily dehydrated to give a trisubstituted olefin. While a wide variety of alcohols can be used, it is preferred to employ secondary alcohols, since these can give reasonably stable ketyls. Further, it is preferred to limit the molecular weight to reduce the size of hetero-coupled products.

In certain embodiments, the chain transfer agent can be added to the hydrolysis reaction.

The molecular weight of product hydrocarbons can be modified by use of metathesis reactions that operate specifically at sites of unsaturation. Olefin metathesis is a process involving the exchange of a bond (or bonds) between similar interacting chemical species such that the bonding affiliations in the products are closely similar or identical to those in the reactants. In such reactions, an olefin described generically as A=A can react with a second olefin, B=B, to yield a cross-over product, A=B. If multiple unsaturated species are available, all possible cross-over products can typically be obtained, with the product ratio determined largely by the concentrations of the reactants. Olefin metathesis of fatty esters has been described in the prior art. See, for example, Mol, J. C.; Buffon, R. J. Braz. Chem. Soc. 1998, 9, 1-11 and Rybak, A.; Fokou, P. A.; Meier, M. A. R. Eur. J. Lipid Sci. Technol. 2008, 110, 797-804. Furthermore, fatty esters can be reacted with ethene to produce product fats with modified properties. This reaction is referred to as ethenolysis. In general, ethenolysis produces compounds with terminal double bonds. In certain embodiments, ethenolysis of fatty oils and triglycerides allows the transformation of long-chain fatty acid triglycerides into fatty oils of lower molecular weight. Such reactions of long chain esters or hydrocarbons with ethene will lead to fuels with 8-14 carbons, which are ideal for kerosene-type fuels.

The metathesis reaction requires a transition metal catalyst. Extensive research has demonstrated that the catalyst may be either heterogeneous or homogeneous with the reaction medium. Common homogeneous catalysts include metal alkylidene complexes as have been described by Schrock, Grubbs, and others. Due to their ease of separation from the reaction products in an industrial scale, and to the lack of a requirement for reactant or product structure specificity, heterogeneous catalysts are preferred in this application. Common heterogeneous metathesis catalysts include rhenium and molybdenum oxides supported on a silica or alumina carrier, and that have been activated with a promoter or co-catalyst. The co-catalyst is typically an alkyl metal compound such as tetrabutyl tin. See, for example, Mandelli, D.; Jannini, M. J. D.; Buffon, R.; Schuchart, U. J. Amer. Oil Chem. Soc. 1996, 73, 229-232.

While the metathesis reaction can be used at any stage in the transformation of triglyceride feedstock into fuel, it is preferred that the metathesis reaction occur prior to acid-catalyzed hydrolysis. The catalysts typically employed for metathesis reactions are sensitive to the presence of hydroxyl functionality, such as would be present in free fatty acids, limiting the reaction to a stage prior to the presence of these groups or after their removal. In certain embodiments, the Kolbe electrolysis gives the highest yield of hetero-coupling products using substrates with 10 or fewer carbons. Performing the metathesis prior to triglyceride hydrolysis will produce esters with intermediate length carbon chains, providing upon hydrolysis an improved substrate for the Kolbe electrolysis.

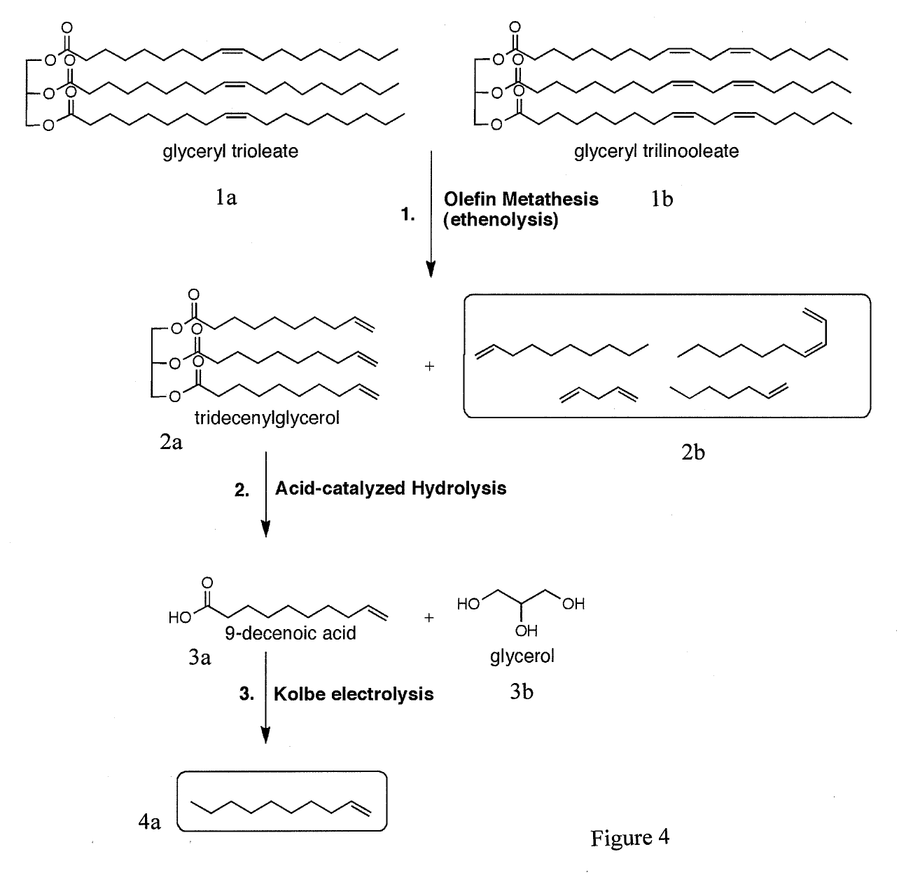

FIG. 4 shows a process for producing hydrocarbon fuel from plant oil in accordance with certain embodiments of the invention. As shown in FIG. 4, glyceryl trioleate 1a and glyceryl trilinooleate 1b are subjected to olefin metathesis (ethenolysis) to produce tridecenylglycerol 2a and a by-product 2b. The tridecenylglycerol 2a is subjected to acid-catalyzed hydrolysis to produce 9-decenoic acid 3a and glycerol 3b. The 9-decenoic acid 3a is subjected to Kolbe electrolysis to produce the linear chain hydrocarbon 4a.

In accordance with certain embodiments of the invention, jatropha oil including triglycerides that contain 44.7% oleic ester, 32.8% linoleic ester, 14.2% palmitic ester, and 7% stearic ester, along with small amounts of myristic, palmitoleic, and linolenic esters can be metathesized with ethene (ethylene) using a catalyst, such as Re₂O₇, supported on silica/alumina with B₂O₃ and tetrabutyl tin as an activator. The reaction can be conducted at a temperature of about 50° C. As a result, a mixture of hydrocarbon products along with glycerol esters with reduced chain lengths can be produced. The mixture can be separated from the heterogeneous catalyst by known conventional techniques, such as by filtration. The filtrate can be treated with water, a Lewis acid catalyst, such as but not limited to zinc oxide, and a phase transfer agent, such as but not limited to tetrabutylammonium chloride, to hydrolyze the esters. The product is a mixture of hydrocarbons and free fatty acids that reflect the composition of the triglyceride feedstock. The fatty acids have some solubility in aqueous media. The protonated acids may be substantially insoluble in the hydrosylate and soluble in the hydrocarbon fraction and therefore, may be easily separated as an oily supernatant.

In certain embodiments, the oily product mixture can be dissolved in isopropanol, and tetrabutylammonium chloride can be added as an electrolyte. The free acids then can be electrolytically oxidized to yield alkane, alkene and mixtures thereof, including 1-octene, 1-nonene, 1-decene, pentadecane, heptadecane, trace amounts of tridecane, 1-heptene, and other hydrocarbons.

In certain other embodiments, the oily product mixture can be dissolved in a mixture of acetic acid, sodium bicarbonate, and ammonium salt electrolyte and electrolytically oxidized to yield 1-octene, 1-nonene, 1-decene, pentadecane, heptadecane, and trace amounts of tridecane, and 1-heptene and other hydrocarbons.

In still certain other embodiments, the oily product mixture can be dissolved in a mixture of acetic acid, isopropanol, sodium bicarbonate, and ammonium salt electrolyte and electrolytically oxidized to yield a complex mixture of 1-octene, 1-nonene, 1-decene, pentadecane, heptadecane, and trace amounts of tridecane, and 1-heptene and other hydrocarbons.

In accordance with certain embodiments of the invention, jatropha oil including triglycerides that contain 44.7% oleic ester, 32.8% linoleic ester, 14.2% palmitic ester, and 7% stearic ester, along with small amounts of myristic, palmitoleic, and linolenic esters can be hydrolyzed with zinc oxide as a Lewis acid catalyst and tetrabutylammonium chloride as a phase transfer agent to give a mixture of free fatty acids that reflect the composition of the triglyceride feedstock. The fatty acids have some solubility in aqueous media, however, the protonated acids may be substantially insoluble in the hydrosylate and therefore, may be easily separated as an oily supernatant.

The oily hydrolysis products can be dissolved in a mixture of acetic acid, sodium bicarbonate, and ammonium salt electrolyte and electrolytically oxidized to yield a mixture of saturated and unsaturated hydrocarbons that can be separated from the electrolyte as low density oil. The oily product can be metathesized using a catalyst, such as but not limited to MoO₃ on silica that has been photoactivated with CO using a mercury lamp and subsequently treated with cyclopropane. The resultant products include 1-octene, 1-nonene, 1-decene, pentadecane, heptadecane, and trace amounts of tridecane, and 1-heptene and other hydrocarbons.

### Example

### Kolbe Electrolysis

To 110 parts decanoic acid in 1340 parts methanol was dissolved 21 parts potassium hydroxide to achieve a pH of about 6. The solution was stirred and treated at room temperature with an electrolytic current of 0.15 amperes at 25 volts. After 10 minutes, the reaction showed complete consumption of decanoic acid and the formation of octadecane as the only product.

To 191 parts of decanoic acid in 1580 parts methanol was dissolved 33 parts potassium hydroxide to achieve a pH of about 6. The solution was stirred and treated at room temperature with an electrolytic current of 0.05 amperes at 6 volts. After 60 minutes, the reaction showed approximately 90% consumption of decanoic acid and the formation of octadecane as the only product.

Hydrolysis

To 15 parts of a solution of 17% water in acetic acid was added 5 parts of waste vegetable oil. The mixture was treated in a microwave reactor at 200° C. at a pressure of 15 bar for 2.5 minutes. 2 parts of water were added to produce a two-phase reaction system with the free fatty acid product isolated from the less dense layer in 95% yield.

Whereas particular embodiments of the invention have been described herein for purposes of illustration, it will be evident to those skilled in the art that numerous variations of the details may be made without departing from the invention as set forth in the appended claims.

What is claimed is:

1. A fuel composition comprising:
a hydrocarbon derived from a biological source selected from the group consisting of plant oil, animal fat and combinations thereof, wherein each of said hydrocarbon and said fuel composition is at least substantially free of oxygen.

2. The fuel composition of claim 1, wherein the biological source is selected from the group consisting of soybean oil, jatropha oil, camelina oil, waste cooking oil, oil from seed crops, and combinations thereof.

3. The fuel composition of claim 1, wherein said hydrocarbon is selected from the group consisting of 1-octene, 1-nonene, 1-decene, pentadecane, heptadecane, tridecane, 1-heptene and mixtures thereof.

4. The fuel composition of claim 1 wherein the biological source comprises triglyceride.

5. The fuel composition of claim 1, wherein said hydrocarbon and said fuel composition have a low cloud point.

6. A method for preparing a fuel composition comprising:
reacting a compound derived from a biological source selected from the group consisting of plant oil, animal fat and combinations thereof, with water to form free fatty acid;
subjecting the free fatty acid to Kolbe electrolysis in the presence of an electrolyte; and
removing an oxygen-containing carboxyl group from the free fatty acid to form a hydrocarbon.

7. The method of claim 6, wherein a chain transfer agent is employed in the Kolbe electrolysis.

8. The method of claim 6, wherein the Kolbe electrolysis is carried out in the presence of a material selected from the group consisting of isopropanol, acetic acid, sodium bicarbonate and mixtures thereof.

9. The method of claim 8, wherein the chain transfer agent comprises isopropanol.

10. The method of claim 6, wherein the electrolyte is selected from the group consisting of tetrabutylammonium chloride, ammonium salt, and mixtures thereof.

11. The method of claim 6, wherein the compound is triglyceride.

12. The method of claim 6, wherein the Kolbe electrolysis comprises reacting said free fatty acid with decanoic acid and acetate in the presence of a solid amine catalyst.

13. The method of claim 6, further comprising conducting olefin metathesis.

14. The method of claim 13, wherein the olefin metathesis is carried out in the presence of ethene.

15. The method of claim 13, wherein the olefin metathesis is conducted in the presence of a catalyst.

16. The method of claim 15, wherein said catalyst is selected from the group consisting of rhenium and molybdenum oxides supported on a carrier selected from the group consisting of silica and alumina, activated with a promoter.

17. The method of claim 13, wherein the olefin metathesis is conducted prior to reacting the compound with water.

18. The method of claim 13, wherein the olefin metathesis is conducted after reacting the compound with water and prior to subjecting the free fatty acid to the Kolbe electrolysis.

19. The method of claim 13, wherein the olefin metathesis is conducted following the removing of the oxygen-containing carboxyl group from the free fatty acid to form hydrocarbon.

20. The method of claim 6, wherein reacting said compound with water is carried out in the presence of a solid catalyst.
